(19) United States
(12) Patent Application Publication
(10) Pub. No.: US 2007/0257050 A1
(43) Pub. Date: Nov. 8, 2007

(54) Blood Glucose Sensor Dispensing Instrument Having a Serrated Knife

(76) Inventors: Mohammad A. Kheiri, Elkhart, IN (US); Russell J. Micinski, South Bend, IN (US)

Correspondence Address: NIXON PEABODY LLP, 161 N. CLARK STREET, 48TH FLOOR, CHICAGO, IL, 60601, US

(21) Appl. No.: 11/662,719

(22) PCT Filed: Sep. 19, 2005
(86) PCT No.: PCT/US05/33290

§ 371 Date: Mar. 13, 2007

Related U.S. Application Data
(60) Provisional application No. 60/611,465, filed on Sep. 20, 2004.

(51) Int. Cl. G01N 33/487

(52) U.S. Cl. 221/2; 221/31

(57) ABSTRACT

A sensor dispensing instrument comprises a housing, a sensor pack, a sensor, and a knife blade assembly. The sensor pack is disposed within the housing and has a sensor cavity and a protective covering. The sensor is disposed within the sensor cavity. The knife blade assembly is operably disposed within the housing and has a serrated edge for cutting the protective covering.

### CROSS-REFERENCE TO RELATED APPLICATIONS

This application claims priority to U.S. Patent Application No. 60/611,465, filed Sep. 20, 2004.

### TECHNICAL FIELD

The present invention relates to a fluid monitoring system, and more particularly, to a new and improved instrument for handling and excising fluid sensors from their packages.

### BACKGROUND

Those who have irregular blood glucose concentration levels are medically required to regularly self-monitor their blood glucose concentration level. An irregular blood glucose level can be brought on by a variety of reasons including an illness such as diabetes. An estimated 18 million people are afflicted with diabetes in the United States alone. A diabetic patient typically monitors their blood glucose concentration level to determine whether the level is too high or too low and whether any corrective action, such as administering insulin or other medication, is necessary to bring the level back within a normal range. The failure to take corrective action can have serious implications. When blood glucose levels drop too low—a condition known as hypoglycemia—a person can become nervous, shaky, and confused. That person's judgment may become impaired and that person may eventually pass out. A person can also become very ill if their blood glucose level becomes too high—a condition known as hyperglycemia. Both hypoglycemia and hyperglycemia can potentially be life-threatening emergencies. As a result, a diabetic may require frequent sampling of his or her blood glucose—typically several times per day.

In one type of blood glucose testing system, sensors are used to test a sample of blood. Such a sensor may have a generally flat, rectangular shape with a front or testing end and a rear or contact end. The sensor contains biosensing or reagent material that will react with blood glucose. The testing end of the sensor is adapted to be placed into the fluid being tested, for example, blood that has accumulated on a person's finger after the finger has been pricked. The fluid is drawn into a capillary channel in the sensor that extends from the testing end to the reagent material. Capillary action causes a sufficient amount of fluid to be drawn into the sensor and into contact with the reagent material. The fluid then chemically reacts with the reagent material in the sensor and generates an electrical signal indicative of the blood glucose level in the blood being tested, which is then supplied to contact areas located near the rear or contact end of the sensor.

In order to couple the electrical signals produced at the sensor contacts to monitoring equipment, the sensors need to be inserted into sensor holders prior to the sensor end being placed into the fluid being tested. The holders have corresponding mating contact areas that become coupled to the contacts on the sensor when the sensor is inserted into the holder. Consequently, the holders act as an interface between the sensor and monitoring equipment that accumulates and/or analyzes the test results.

Prior to being used, the sensors need to be maintained at an appropriate humidity level so as to insure the integrity of the reagent materials in the sensor. For example, sensors may be packaged individually in tear-away packages that are designed to maintain a proper humidity level. Blister type packaging methods are particularly suitable for this application. In this regard, the packages can include desiccant material to maintain the proper humidity inside the package. In order for a person to use an individual sensor for testing blood glucose, the package must be opened by tearing the seal. Alternatively, some packages require the user to exert force against one side of the package resulting in the sensor bursting or rupturing the foil on the other side. As can be appreciated, the opening of these packages can be difficult. Moreover, once the package is opened, the user needs to be sure that the sensor is not damaged or contaminated as it is being placed into the sensor holder and used to test the blood sample.

U.S. Pat. No. 5,630,986, issued on May 20, 1997, and entitled Dispensing Instrument For Fluid Monitoring Sensors (referred to hereinafter as “the '986 patent”), discloses a type of sensor pack with multiple sensors and a testing blood glucose and dispensing instrument for handling the sensors contained in the sensor pack. In particular, the sensor dispensing instrument disclosed in the '986 patent is adapted to receive a sensor pack containing a plurality of blood glucose sensors. The sensor pack includes a circular base having a plurality of sensor retaining cavities, each of which hold an individual sensor, and a foil covering which encloses a sensor inside each of the cavities. Each of the sensors has a generally flat, rectangular shape with a front testing end through which fluid is drawn so as to react with a reagent material in the sensor and an opposite, rear contact end.

The sensor instrument disclosed in the '986 patent includes an outer housing having an upper and a lower case that are pivotable with respect to each other so that the sensor pack can be positioned on an indexing disk disposed inside the housing. Two slides on the upper case of the housing (a slide latch and a separate slide actuator) control whether the instrument is in a display mode or in a testing mode. The instrument is placed into its display mode by moving the slide latch laterally and pushing the slide actuator away from a standby position. When in the display mode, a person using the instrument can view data displayed on a display unit in the upper case and/or input data into the instrument.

The instrument is in its testing mode when the slide latch is in its normal position and the slide actuator is pushed towards its testing position. As the slide actuator is moved towards its actuated position, the driver with the knife blade thereon moves toward the testing position of the feed mechanism as the disk drive arm travels in a straight, radially extending groove in the indexing disk (which prevents the disk from rotating). At the same time, the knife blade is moved towards one of the sensor cavities in the sensor pack such that a beveled edge of the knife blade pierces the foil covering the sensor cavity to engage the sensor disposed in the cavity. As the slide actuator and the driver are pushed further toward the actuated position of the actuator, the knife blade ejects the sensor out from the sensor cavity and into a testing position near the testing end of the sensor housing.

Once the blood analyzing test is completed, the slide actuator is moved in the opposite direction towards its standby position so that the sensor can be removed from the dispensing instrument. The continued retraction of the driver causes the indexing disk drive arm to travel along a curvilinearly extending groove in the indexing disk, resulting in the rotation of the indexing disk. The rotation of the indexing disk results in the sensor pack being rotated so that the next sensor is positioned in alignment with the knife blade for the next blood glucose test that is to be performed.

Although the sensor instrument disclosed in the 1986 patent overcomes many of the problems discussed above in connection with the use of individual sensors, in some instances, the straight, beveled edge of the knife blade will tear or rip the foil rather than cutting it. This may cause shards of foil to dislodge and possibly get caught in the nose of the instrument, which can cause jamming of the sensor. Although tearing and dislodging of the foil is a relatively rare occurrence, given the frequency with which a diabetic must test their blood glucose level, the possibility of jamming a sensor with a shard of foil must be considered as a potential problem. What is needed is a sensor instrument with an improved knife that reduces the frequency of ripped or dislodged packaging material.

### BRIEF SUMMARY

Accordingly, an object of the present invention is to provide a new and improved sensor dispensing instrument for handling the sensors contained in a sensor pack and used for testing blood glucose. In particular, objects of the present invention are to provide a new and improved fluid sensor dispensing instrument handling device having a serrated knife that reduces the possibility of jams, and which otherwise overcomes the problems or limitations discussed above.

In accordance with these and many other objects of the present invention, the present invention is embodied in a sensor dispensing instrument comprising a housing, a sensor pack, a sensor, and a knife blade assembly. The sensor pack is disposed within the housing and has a sensor cavity and a protective covering. The sensor is disposed within the sensor cavity. The knife blade assembly is operably disposed within the housing and has a serrated edge for cutting the protective covering.

According to a second aspect of the present invention, a sensor dispensing instrument is configured to be used with a sensor pack containing a plurality of sensors. Each of the plurality of sensors is disposed in a sensor cavity on the sensor pack and enclosed by a protective covering. The sensor dispensing instrument is further adapted to perform a test using one of the plurality of sensors, and comprises a knife blade assembly. The knife blade assembly has a serrated edge for puncturing and cutting the protective covering and ejecting one of the sensors from the sensor cavity.

A third aspect of the present invention is embodied in a method of making a knife assembly for a sensor dispensing instrument. The method includes the acts of providing a metal sheet and fabricating a knife from the metal sheet. The method also includes the act of etching a serrated cutting edge on the knife. The method also includes the act of molding a plastic body around the knife.

### BRIEF DESCRIPTION OF THE DRAWINGS

FIG. 1 is a top perspective view of a blood glucose sensor dispensing instrument embodying the present invention.

FIG. 2 is an exploded perspective view of component parts of the sensor dispensing instrument of FIG. 1.

FIG. 3 is an exploded perspective view of a sensor pack used in the blood glucose sensor dispensing instrument of FIG. 1 with the foil portion of the sensor pack separated from the base portion of the sensor pack.

FIG. 4 is a perspective view of the knife assembly and sensor pack used in the blood glucose sensor dispensing instrument of FIG. 1.

FIG. 5 is a perspective view of the knife assembly used in the blood glucose sensor dispensing instrument of FIG. 1.

FIG. 6 is an end view of the knife assembly used in the blood glucose sensor dispensing instrument of FIG. 1.

FIG. 7 is a side view of the knife assembly used in the blood glucose sensor dispensing instrument of FIG. 1.

FIG. 8 is a top view of the knife assembly used in the blood glucose sensor dispensing instrument of FIG. 1.

FIG. 9 is a detail view of the serrated edge of the knife assembly of FIG. 7.

### DETAILED DESCRIPTION OF THE DRAWINGS AND THE PRESENTLY PREFERRED EMBODIMENTS

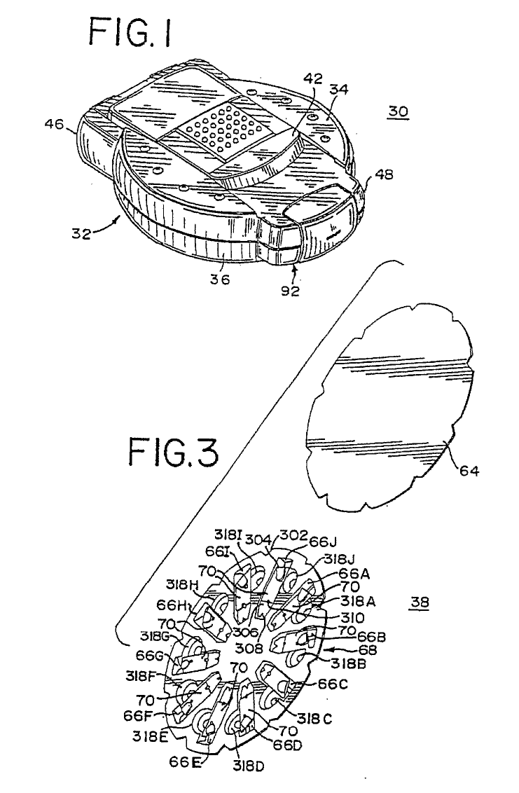

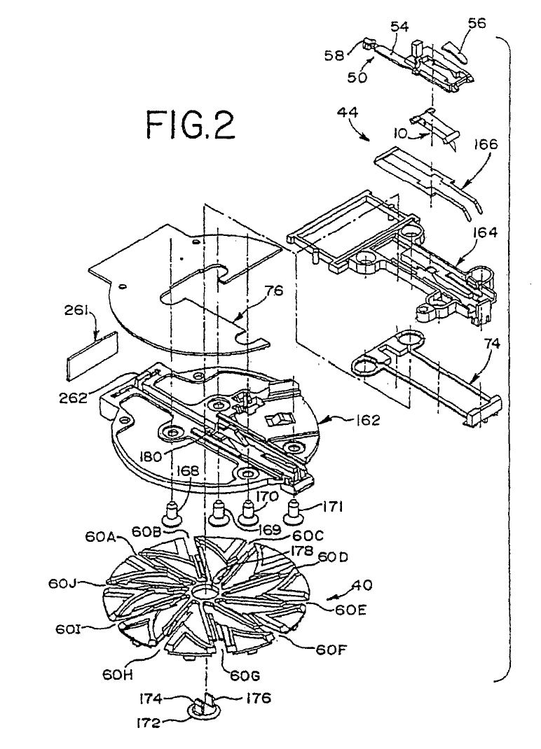

Referring now more specifically to the drawings, a blood glucose sensor dispensing instrument generally designated by the reference numeral 30 and embodying the present invention is shown in FIG. 1. The sensor dispensing instrument 30 includes an outer housing 32 having an upper case 34 and a lower case 36, a display (not shown), and an electronics assembly (not shown). The upper case 34 is pivotable with respect to the lower case 36 in a clam shell fashion so that a sensor pack 38 (FIG. 3) can be positioned on an indexing disk 40 (FIG. 2) within the housing 32. With the sensor pack 38 so loaded in the housing 32, a slide actuator 42 on the upper case 34 of the housing 32 can be moved to engage a feed mechanism, generally designated by the numeral 44 (FIG. 2), secured to the inside of the upper case 34. The slide actuator 42 can be manually pushed from a standby position (FIG. 1) adjacent a rear end 46 of the upper case 34 toward an actuated or testing position (not shown) adjacent a forward or testing end 48 of the upper case 34. The slide actuator 42 also can be moved to place the sensor dispensing instrument 30 in a data processing or display mode.

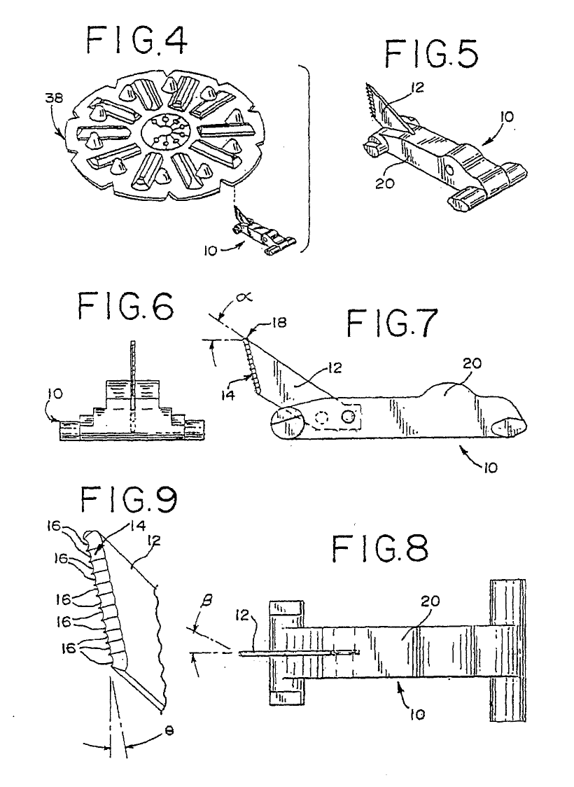

As seen in FIG. 2, the feed mechanism 44 includes a driver 50, a knife blade assembly 10, and a knife spring 56. The knife blade assembly 10 is pivotally mounted on driver 50. Illustrated in FIGS. 4-9, one embodiment of a knife blade assembly 1° comprises a blade 12 and a plastic body 20. The blade 12 may be insert molded into plastic body 20, with the blade 12 placed into a mold and the plastic body 20 molded over it. Other methods of attaching blade 12 to plastic body 20 may also be used, such as an adhesive or friction fit. Plastic body 20 may be formed from a polycarbonate compound, such as LNP DL 4020 SM (available from LNP Engineering Plastics, located in Exton Pa., USA) or RTP 399×97886 (available from RTP Company, located in Winona, Minn., USA), 10% TEFLON™ (tetrafluoroethylene or TFE) filled or 1% Fluoroguard filled polycarbonate, although other materials may be used.

In the embodiment shown in FIGS. 4-9, blade 12 is formed from a chemical etching process, although other sheet metal fabrication techniques such as stamping or laser cutting may also be used. In the embodiment illustrated, blade 12 is formed from 0.010 inch 302 Stainless Steel Stock, tempered half to three quarters hard. As seen in FIG. 9, blade 12 has a serrated edge 14. Serrated edge 14 may be formed by the same chemical etching process used to form blade 12, described above, to form a clean and consistent cutting edge. Serrated edge 14 is formed from a plurality of curved segments 16 that are arcuate and convex in shape and intersect one another at an angle. Curved segments 16 preferably are formed with a radius of 0.006 inches, although other shapes and sizes may be used. This arrangement increases the length of the cutting edge on serrated edge 14. In the embodiment shown in FIG. 9, serrated edge 14 has eleven curved segments 16, although any number of segments may be used to form serrated edge 14. Further, the embodiment shown in FIG. 9 uses a two-dimensional chemical etching process, forming serrated edge 14 only on one side of blade 12. The other side of blade 12 may also be serrated, for example, by a three dimensional chemical etching process or a separate etching on the other side.

As serrated edge 14 of knife assembly 10 must first pierce then cut a foil covering 64 on a sensor pack 38 (as shown in FIG. 3 and described below), a top surface 18 of blade 12 is formed at an angle a of approximately 32.5 degrees relative to a horizontal line, as seen in FIG. 7. Serrated edge is formed at angle e of approximately 14 degrees relative to a vertical line, as seen in FIG. 9. As seen in FIG. 8, serrated edge 14 has a cutting angle 13 of approximately 30-45 degrees, although other angles may be used.

Referring back to FIG. 2, an indexing disk drive arm 54 on which is mounted the knife spring 56 extends from the feed mechanism 44. In operation, as the slide actuator 32 is moved toward its actuated position, the driver 50 with the knife blade assembly 10 thereon moves toward the testing end 48 (FIG. 1) and a button 58 at the distal end of the disk drive arm 54 travels in one of a plurality of radially extending grooves 60A-J in the indexing disk 40 such that the indexing disk 40 is not rotated while the driver 50 is being moved towards its testing position. As the knife blade assembly 10 is being moved toward the testing end 48, top surface 18 of the serrated edge 14 on the knife blade assembly 10 pierces a portion of foil 64 covering one of a plurality of sensor cavities 66A-J (seen in FIG. 3), such as the cavity 66F, in a base portion 68 of the sensor pack 38 in alignment with the knife blade 62. A sensor 70 disposed in the cavity 66A is engaged by the serrated edge 14 resulting in the serrated edge 14 further severing the foil 64 covering the sensor cavity 66F and forcing or ejecting the sensor 70 out from the sensor cavity 66F.

A block guide 162, a printed circuit board 76, a sensor actuator 74, a housing guide 164, a second knife spring 166, the knife blade assembly 10, and the driver 50 are held together and secured to the upper case 34 by fasteners 168-171 that extend through those components and into the upper case 34 (FIG. 2). In addition, the indexing disk 40 is secured relative to the upper case 34 by being rotatably retained on the housing guide 164 by a retainer 172 that has a pair of latch arms 174 and 176 extending through a central hole 178 in the indexing disk 40 and latching into an opening 180 in the block guide 162. An elastomeric connector 261 couples calibration circuitry (not shown) to circuitry on the printed circuit board 76. Connector 261 is disposed in a channel 262 located in the block guide 162. Additional details regarding the device shown in FIGS. 1-2 are described in the '986 patent. It should be noted that the sensor dispensing instrument 30 of the present invention incorporates additional components that are similar in design and/or function as those described in the '986 patent. The contents of this patent are hereby incorporated by reference to avoid the unnecessary duplication of the description of these additional components. Examples of sensor dispensing instruments are the Ascensia® BREEZE™ Blood Glucose Monitoring System or the Ascensia® DEX® 2 Blood Glucose Monitoring System, available from Bayer Corporation of Elkhart, Ind., United States of America.

Similarly, the sensor pack 38 (shown in FIG. 3) utilized by the sensor dispensing instrument 30 is of the type described in U.S. Pat. No. 5,575,403, issued Nov. 19, 1996, and entitled Dispensing Instrument For Fluid Monitoring Sensors, the contents of which are hereby incorporated by reference. One example of such a sensor pack is the Ascensia® AUTODISC™ with ten test strips also available from Bayer Corporation of Elkhart, Ind., United States of America. In general, the sensor pack 38 is adapted to house ten sensors 70, with one of the ten sensors 70 in each of ten separate sensor cavities 66A-J. Each of the sensors 70 has a generally flat, rectangular shape extending from a front or testing end 304 to a back end 306. The front end 304 is angled so that it will puncture an unsevered portion of the protective foil 64 overlying the sensor cavity 66J as the sensor 70 is being forced out of the sensor cavity 66J by serrated edge 14. The front end 304 is also adapted to be placed into blood that is being analyzed. The back end 306 of the sensor 70 includes a small notch 308 that is engaged by the knife blade assembly 10 as the knife blade assembly 10 ejects the sensor 70 from the sensor cavity 66J. Contacts (not shown) near the back end 306 of the sensor 70 are adapted to mate with metal contacts (not shown) on a sensor actuator (not shown) when the sensor 70 is in the testing position. As a result, the sensor 70 is coupled to the electronic circuitry on the circuit board assembly 42 so that information generated in the sensor 70 during testing can be stored, analyzed and/or displayed.

The sensor pack 38 is formed of the circularly shaped base portion 68 and the correspondingly configured foil 64. The sensor cavities 66A-J are formed as depressions in the base portion 68 with each of the sensor cavities 66A-J adapted to house one of the sensors 70. The foil 64 is adapted to cover the top of the base portion 68 and be affixed to the base portion by heat sealing along the entire outer peripheral edge of the foil 64 to the outer peripheral edge of the base portion 68. The foil 64 also is heat sealed about the entire perimeter of each set of the sensor retaining cavities 66A-J and the desiccant cavities 318A-J to seal the sensor retaining cavities 66A-J and the desiccant cavities 318A-J such that the individual sensors 70 are maintained in a desiccated state and isolated from each other. As a result, the opening of one of the sensor cavities 66A-J will not affect the desiccated state of any of the other sensor cavities 66A-J. The foil 64 may be made of any material that will adequately seal the sensor cavities 66A-J and the desiccant cavities 318A-J while providing a material that may be readily severed by the knife blade assembly 10 and pierced by the sensor 70 as it is being pushed out from the sensor cavities 66A-J. One type of foil that can be used for the foil 64 is AL-191-01 foil distributed by Alusuisse Flexible Packaging, Inc.

Thus, there has been disclosed in accordance with the present invention, a blood glucose sensor dispensing instrument with a knife assembly having a cleaner and more consistent cutting edge than a straight blade. With a serrated cutting edge, the present invention more easily and cleanly pierces and cuts a foil cover on a sensor pack. This improves the reliability of the cut, reducing the frequency with which foil covers on sensor packs are torn, ripped or dislodged. In turn, the number of sensors jammed with a shard of foil is reduced. Moreover, the serrated cutting edge may improve engagement with the sensor, allowing for a sensor to be ejected more cleanly through the foil cover. The serrated cutting edge also allows for a reduced cost—allowing for a single process to form both the blade and the serrated edge, in contrast to separate stamping and grinding operations. Further, the serrated edge increases the useful life of the sensor dispensing instrument.

### Alternative Embodiment A

A sensor dispensing instrument comprising:

a housing;

a sensor pack disposed within said housing having a sensor cavity and a protective covering;

a sensor disposed within said sensor cavity; and

a knife blade assembly operably disposed within said housing and having a serrated edge for cutting said protective covering.

### Alternative Embodiment B

The sensor dispensing instrument of embodiment A wherein said knife blade assembly is operably disposed to puncture said protective covering and eject said sensor from said sensor cavity.

### Alternative Embodiment C

The sensor dispensing instrument of embodiment A wherein said protective covering is foil.

### Alternative Embodiment D

The sensor dispensing instrument of embodiment A wherein said serrated edge has a plurality of curved segments.

### Alternative Embodiment E

The sensor dispensing instrument of embodiment A wherein said knife blade assembly further comprises a plastic body and a blade fastened to said body.

### Alternative Embodiment F

The sensor dispensing instrument of embodiment E wherein said blade is insert molded into said body.

### Alternative Embodiment G

The sensor dispensing instrument of embodiment A wherein said sensor pack has a circular shape and a plurality of circumferentially arranged sensor cavities.

### Alternative Embodiment H

A sensor dispensing instrument configured to be used with a sensor pack containing a plurality of sensors, each of said plurality of sensors being disposed in a sensor cavity on said sensor pack and enclosed by a protective covering, said sensor dispensing instrument further adapted to perform a test using one of said plurality of sensors, and comprising a knife blade assembly having a serrated edge for puncturing and cutting said protective covering and ejecting one of said sensors from said sensor cavity.

### Alternative Embodiment I

The sensor dispensing instrument of embodiment H wherein said protective covering is foil.

### Alternative Embodiment J

The sensor dispensing instrument of embodiment H wherein said sensor pack has a circular shape and a plurality of circumferentially arranged sensor cavities.

### Alternative Embodiment K

The sensor dispensing instrument of embodiment H wherein said serrated edge has a plurality of curved segments.

### Alternative Embodiment L

The sensor dispensing instrument of embodiment H wherein said knife blade assembly further comprises a plastic body and a blade fastened to said body.

### Alternative Embodiment M

The sensor dispensing instrument of embodiment H wherein said blade is insert molded into said body.

### Alternative Process N

A method of making a knife assembly for a sensor dispensing instrument, the method comprising the acts of:

providing a metal sheet;

fabricating a knife from said metal sheet;

etching a serrated cutting edge on said knife; and

molding a plastic body around said knife.

### Alternative Process O

The method of process N wherein said step of fabricating a knife from said metal sheet is performed by chemical etching.

### Alternative Process P

The method of process N wherein said serrated cutting edge has a plurality of curved segments.

### Alternative Process Q

The method of process N wherein said knife assembly is assembled into a sensor dispensing instrument.

Although the invention has been described and illustrated with reference to specific illustrative embodiments thereof, it is not intended that the invention be limited to those illustrative embodiments. Those skilled in the art will recognize that variations and modifications can be made without departing from the spirit of the invention. For example, whereas the serrated cutting edge is disclosed as being fabricated from stainless steel, other materials such as plastic, ceramic, or other metals may also be used. Alternately, the shape of the serrations may also be modified from the curved segments disclosed above to triangular, convex, or other shaped teeth. Moreover, an additional cutting edge on the other side of the knife may be added, or more than one blade may be used. Further, the angles at which the blade is formed may be altered, and a different cutting angle may be used. It is therefore intended to include within the invention all such variations and modifications that fall within the scope of the appended claims and equivalents thereof.

1. A sensor dispensing instrument comprising:
a housing;
a sensor pack disposed within said housing having a sensor cavity and a protective covering;
a sensor disposed within said sensor cavity; and
a knife blade assembly operably disposed within said housing and having a serrated edge for cutting said protective covering.

2. The sensor dispensing instrument of claim 1, wherein said knife blade assembly is operably disposed to puncture said protective covering and eject said sensor from said sensor cavity.

3. The sensor dispensing instrument of claim 1, wherein said protective covering is foil.

4. The sensor dispensing instrument of claim 1, wherein said serrated edge has a plurality of curved segments.

5. The sensor dispensing instrument of claim 1, wherein said knife blade assembly further comprises a plastic body and a blade fastened to said body.

6. The sensor dispensing instrument of claim 5, wherein said blade is insert molded into said body.

7. The sensor dispensing instrument of claim 1, wherein said sensor pack has a circular shape and a plurality of circumferentially arranged sensor cavities.

8. A sensor dispensing instrument configured to be used with a sensor pack containing a plurality of sensors, each of said plurality of sensors being disposed in a sensor cavity on said sensor pack and enclosed by a protective covering, said sensor dispensing instrument further adapted to perform a test using one of said plurality of sensors, and comprising a knife blade assembly having a serrated edge for puncturing and cutting said protective covering and ejecting one of said sensors from said sensor cavity.

9. The sensor dispensing instrument of claim 8, wherein said protective covering is foil.

10. The sensor dispensing instrument of claim 8, wherein said sensor pack has a circular shape and a plurality of circumferentially arranged sensor cavities.

11. The sensor dispensing instrument of claim 8, wherein said serrated edge has a plurality of curved segments.

12. The sensor dispensing instrument of claim 8, wherein said knife blade assembly further comprises a plastic body and a blade fastened to said body.

13. The sensor dispensing instrument of claim 8, wherein said blade is insert molded into said body.

14. A method of making a knife assembly for a sensor dispensing instrument, the method comprising the acts of:
providing a metal sheet;
fabricating a knife from said metal sheet;
etching a serrated cutting edge on said knife; and
molding a plastic body around said knife.

15. The method of claim 14, wherein said step of fabricating a knife from said metal sheet is performed by chemical etching.

16. The method of claim 14, wherein said serrated cutting edge has a plurality of curved segments.

17. The method of claim 14, wherein said knife assembly is assembled into a sensor dispensing instrument.
